(19) United States
(12) Patent Application Publication
(10) Pub. No.: US 2023/0400473 A1
(43) Pub. Date: Dec. 14, 2023

(54) METHODS AND COMPOSITIONS FOR THE TREATMENT OF CROHN'S DISEASE

(71) Applicant: Children's Hospital Medical Center, Cincinnati, OH (US)

(72) Inventors: Phillip Minar, Montgomery, OH (US); Anil G. Jegga, West Chester, OH (US)

(21) Appl. No.: 18/207,707

(22) Filed: Jun. 9, 2023

Related U.S. Application Data
(60) Provisional application No. 63/350,937, filed on Jun. 10, 2022.

(51) Int. Cl. G01N 33/68

(52) U.S. Cl. CPC G01N 33/6893; G01N 2800/065; G01N 2800/52; G01N 2333/9123; G01N 2333/70578; G01N 2333/96433; G01N 2333/4713; G01N 2333/70535

(57) ABSTRACT

Disclosed herein anti-TNF therapy companion diagnostics (e.g., a predictive biomarker panel) for management of Crohn's Disease (CD). The disclosed companion diagnostics may be used to identify an appropriate treatment for a patient, and includes, for example, in vitro diagnostic tests or devices that provide information for the use of an anti-TNF therapy. The disclosed methods may be used, in certain aspects, for the identification of patients likely to respond, or as not likely to respond to an anti-TNF agent. The use of the disclosed methods may allow for dose determination, discontinuation, or the administration of combinations of therapeutic agents.

### CROSS-REFERENCE TO RELATED APPLICATIONS

This application claims priority to and benefit of U.S. provisional application Ser. No. 63/350,937, filed Jun. 10, 2023, the contents of which are incorporated in their entirety for all purposes.

### STATEMENT REGARDING FEDERALLY-SPONSORED RESEARCH

This invention was made with government support under DK105229 and DK118314 awarded by the National Institutes of Health. The government has certain rights in the invention.

### BACKGROUND

Despite the rapid expansion of the gastroenterologists' therapeutic armory of biologic and small molecules to manage patients with inflammatory bowel disease (IBD), biologics targeting tumor necrosis factor-α (anti-TNF) have remained first-line therapy for children with moderate to severe Crohn's disease (CD) in North America. Early use of “top-down” anti-TNF therapy has led to reduced rates of penetrating complications, reversal of growth failure, and a monumental decline in corticosteroid exposures to manage gastrointestinal symptoms. However, with an anti-TNF primary non-response rate of 20% and a secondary nonresponse rate of 25-40% unrelated to subtherapeutic drug concentrations, there is a critical need to better define the anti-TNF response CD subsets with immune signatures to pair the right biologic for the individual patient. Early use of “top-down” anti-TNF therapy in pediatric CD has led to reversal of growth failure, decline in corticosteroid exposures and improved quality of life and is associated with a reduction in penetrating complications and a favorable safety profile. With an anti-TNF primary non-response rate of 20%, a secondary nonresponse rate of 25-40% unrelated to drug exposure and novel therapies available, there is a critical need to better prognosticate anti-TNF response to specific CD subsets.

### BRIEF SUMMARY

Disclosed herein anti-TNF therapy companion diagnostics (e.g., a predictive biomarker panel) for management of Crohn's Disease (CD). The disclosed companion diagnostics may be used to identify an appropriate treatment for a patient, and includes, for example, in vitro diagnostic tests or devices that provide information for the use of an anti-TNF therapy. The disclosed methods may be used, in certain aspects, for the identification of patients likely to respond, or as not likely to respond to an anti-TNF agent. The use of the disclosed methods may allow for dose determination, discontinuation, or the administration of combinations of therapeutic agents.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 depicts overlaps among the different candidate modules across three analyses.

FIG. 2 is an annotated heatmap showing gene expression in various modules.

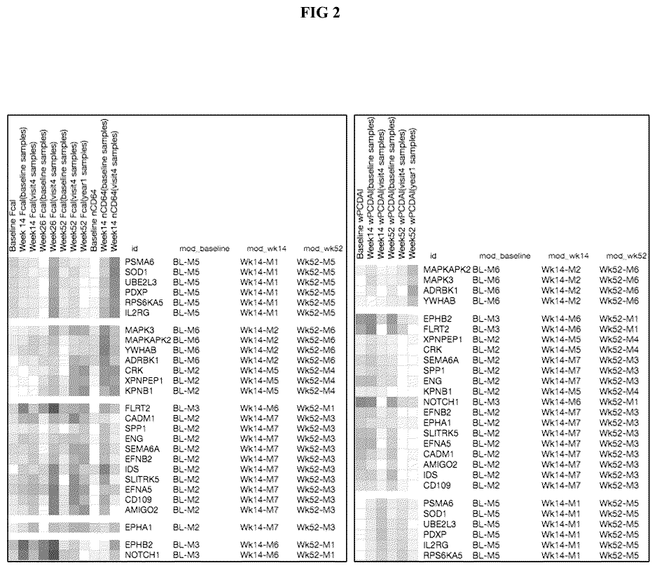

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

The methods may comprise, consist of, or consist essentially of the elements of the compositions and/or methods as described herein, as well as any additional or optional element described herein or otherwise useful in methods for treating intestinal inflammation and reducing Crohn's Disease (CD)-related complications in pediatric IBD patients in an individual in need thereof, or related compositions for carrying out the disclosed methods. The disclosed methods may be performed outside the body of a subject (ex vivo, for example in vitro).

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

The term biomarker includes, but is not limited to, genetic regulation, protein levels, RNA levels, blood and/or tissue cultures, and cellular responses.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

“Sequence identity” as used herein indicates a nucleic acid sequence that has the same nucleic acid sequence as a reference sequence, or has a specified percentage of nucleotides that are the same at the corresponding location within a reference sequence when the two sequences are optimally aligned. For example a nucleic acid sequence may have at least 50%, 60%, 70%, 75%, 80%, 85%, 90%, 95%, 96%, 97%, 98%, 99%, or 100% identity to the reference nucleic acid sequence. The length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides, and most preferably the full length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

The combination of a rising incidence of younger children diagnosed with CD and the high rate of CD-related complications in children has led to a paradigm shift to tailor biologic therapy much earlier than previous treatment algorithms recommended. Infliximab is a recombinant chimeric immunoglobulin (Ig)G1 monoclonal antibody (mAb) that neutralizes the biologic activity of soluble and membrane-bound tumor necrosis factor-α (TNF) and is conventionally dosed at 5 mg/kg at 0, 2, and 6 weeks during induction followed by infusions every 8 weeks during maintenance. The RISK study (an inception cohort of 913 pediatric onset CD) found that early use (within 90 days from diagnosis) of anti-TNF therapies prevented penetrating, but not stricturing, complications. While studies have shown variable response rates of intestinal healing are likely to improve with optimization of anti-TNF dosing regimens, and effective dose optimization strategies following proactive therapeutic drug monitoring, a sizable percentage of children will continue to fail anti-TNF despite adequate drug exposure. Although anti-TNF therapies target a significant proinflammatory cytokine in CD, there are no current objective patient biomarkers that allow clinicians to reliably predict which patients are most likely to respond to infliximab.

Disclosed herein anti-TNF therapy companion diagnostics (e.g., a predictive biomarker panel) for management of Crohn's Disease (CD). The disclosed companion diagnostics may be used to identify an appropriate treatment for a patient, and includes, for example, in vitro diagnostic tests or devices that provide information for the use of an anti-TNF therapy. The disclosed methods may be used, in certain aspects, for the identification of patients likely to respond, or as not likely to respond to an anti-TNF agent. The use of the disclosed methods may allow for dose determination, discontinuation, or the administration of combinations of therapeutic agents.

Disclosed herein are methods for treating intestinal inflammation and/or reducing Crohn's Disease (CD)-related complications in an individual in need thereof. In one aspect, the method may comprise
detecting one or more biomarker of a predictive biomarker panel in a biological sample of the individual;
determining whether the individual is likely to have a therapeutic response to an anti-TNF treatment based on the detecting of one or more biomarker; and
administering one or both of an increased dosage of anti-TNF treatment and a combination therapy that includes an anti-TNF treatment to the individual determined to have the therapeutic response to said anti-TNF treatment.

In one aspect, the biomarker may be selected from FCN3, CKM, SOST, TNFRSF11A, PRSS27, HBA1, FCN3, SH2D1A, TNFRSF11A, EPB41, and combinations thereof. The full sequences and variants will be appreciated by one of ordinary skill in the art with reference to typical databases such as those available within ncbi.nlm.gov and the supporting publications. The biomarker detected may be in the form of a protein, a peptide, a variant, or a fragment thereof. In one aspect, the variant or fragment may contain one or more conserved regions of the biomarker. In further aspects, the detection of the biomarker may be carried out via detection of expression of the biomarker, via detection of, and optionally quantification of, mRNA encoding the biomarker.

In one aspect, the one or more biomarker may be selected from ENG, CADM1, EFNA5, AMIGO2, SEMA6A, EFNB2, EPHA1, SPP1, SLITRK5, CD109, IDS, FLRT2, NOTCH1, EPHB2, and combinations thereof, wherein a deviation from a control value for the biomarker indicates that the individual is a responder.

In one aspect, the one or more biomarker may be selected from MAPK3, YWHAB, ADRBK1, MAPKAPK2, CRK, KPNB1, XPNPEP1, UBE2L3, PDXP, RPS6KA5, PSMA6, IL2RG, SOD1, and combinations thereof, wherein a deviation from a control value for the biomarker indicates that the individual is a non-responder.

In one aspect, the one or more biomarker may comprise a biomarker panel. In this aspect, the biomarker panel may comprise from about 5 to about 20 protein biomarkers, or from about 10 to about 15 protein biomarkers, selected from ENG, CADM1, EFNA5, AMIGO2, SEMA6A, EFNB2, EPHA1, SPP1, SLITRK5, CD109, IDS, FLRT2, NOTCH1, EPHB2, MAPK3, YWHAB, ADRBK1, MAPKAPK2, CRK, KPNB1, XPNPEP1, UBEL2L3, PDXP, RPS6KA5, PSMA6, IL2RG, and SOD1.

In one aspect, the detecting may be carried out following an initial treatment of the individual. For example, the detecting may be carried out by week 1 of the treatment, by week 2 of the treatment, by week 3 of the treatment, by week 4 of the treatment, by week 5 of the treatment, by week 6 of the treatment, by week 7 of the treatment, by week 8 of the treatment, by week 9 of the treatment, by week 10 of the treatment, by week 11 of the treatment, by week 12 of the treatment, by week 13 of the treatment, by week 14 of treatment, by week 15 of the treatment, by week 16 of the treatment, by week 17 of the treatment, by week 18 of the treatment, by week 19 of the treatment, or by week 20 of the treatment.

In one aspect, the one or more biomarker may comprise a first biomarker and a second biomarker, wherein the first biomarker is CD64.

In one aspect, the anti-TNF treatment may be selected from infliximab, adalimumab, and combinations thereof.

The biological sample may be any in which the biomarker may be detected. For example, in various aspects, the biological sample may be selected from plasma, blood (venous or arterial), serum, urine, saliva, cerebrospinal fluid (CSF), synovial fluid, amniotic fluid, breast milk, sweat (eccrine or apocrine), nasal secretions, feces (stool), a tissue sample (e.g. bone marrow), or a combination thereof. In one aspect, the biological sample is a plasma sample. The detecting may be carried out at a single time point, at two time points, at three time points, at four time points, or at more than four time points. The time points may include prior to administration or after administration of therapy. In one aspect, the individual is anti-TNF naïve, meaning that the individual has not yet received an anti-TNF therapy when the detection is carried out. The interval between the time points may be at least one day, at least two days, at least three days, at least four days, at least five days, at least six days, at least seven days, or every two weeks, or every three weeks, or monthly.

In one aspect, the determining may comprise detecting a one-fold change, or a two-fold change, or more than two-fold change, in said biomarker as compared to a control value. The control value may be, for example, a level in a healthy control or a baseline level in said individual. The control value may be a value prior to treatment in the individual, or an average of values over time in the individual prior to treatment, or the averages of values over time of an individual having a known status, such as that of a healthy control or a known responder or nonresponder.

In one aspect, the detecting may be carried out via methods known in the art, for example, an immunoassay such as ELISA (enzyme linked immunosorbent assay) or RIA (radioimmunoassay) for protein, or PCR (polymerase chain reaction) for nucleotide detection. Methods of detecting the biomarkers will be readily appreciated by one of ordinary skill in the art.

In one aspect, a plurality of detection agents for use as a companion diagnostic is disclosed. In one aspect, the plurality of detection agents may comprise at least 2, or at least 3 or a least 4, or at least 5, or at least 6, or at least 7, or at least 8, or at least 9, or at least 10, or at least 11, or at least 12, or at least 13, or at least 14, or at least 15, or at least 16, or at least 17, or at least 18, or at least 19, or at least 20 detection agents, each detection agent being specific for a biomarker, the biomarker being selected from ENG, CADM1, EFNA5, AMIGO2, SEMA6A, EFNB2, EPHA1, SPP1, SLITRK5, CD109, IDS, FLRT2, NOTCH1, EPHB2, MAPK3, YWHAB, ADRBK1, MAPKAPK2, CRK, KPNB1, XPNPEP1, UBEL2L3, PDXP, RPS6KA5, PSMA6, IL2RG, and SOD1.

In one aspect, the plurality of detection agents may be antibodies specific to said at least one biomarker, or at least two biomarkers, or at least two biomarkers, or at least three biomarkers, or at least four biomarkers, or at least five biomarkers, or at least six biomarkers, or at least seven biomarkers, or at least eight biomarkers, or at least nine biomarkers, or at least ten biomarkers, or at least 12 biomarkers, or at least 13 biomarkers, or at least 14 biomarkers, or at least 15 biomarkers, or at least 16 biomarkers, or at least 17 biomarkers, or at least 18 biomarkers, or at least 19 biomarkers, or at least 20 biomarkers as disclosed herein.

In one aspect, the plurality of detection agents may comprise nucleic acids specific to at least one gene expressing at least one biomarker, at least two genes expressing at least two biomarkers, at least three genes expressing at least three biomarkers, at least four genes expressing at least four biomarkers, at least five genes expressing at least five biomarkers, at least six genes expressing at least six biomarkers, at least seven genes expressing at least seven biomarkers, at least eight genes expressing at least eight biomarkers, at least nine genes expressing at least nine biomarkers, at least ten genes expressing at least ten biomarkers, at least 11 genes expressing at least 11 biomarkers, at least 12 genes expressing at least 12 biomarkers, at least 13 genes expressing at least 13 biomarkers, at least 14 genes expressing at least 14 biomarkers, at least 15 genes expressing at least 15 biomarkers, at least 16 genes expressing at least 16 biomarkers, at least 17 genes expressing at least 17 biomarkers, at least 18 genes expressing at least 18 biomarkers, at least 19 genes expressing at least 19 biomarkers, or at least 20 genes expressing at least 20 biomarkers.

In one aspect, the detection agent may comprise a label for detection. Suitable labels will be known in the art, and include, for example, radiolabels or fluorescent labels. In one aspect, the label is capable of quantification.

In one aspect, the plurality of detection agents is provided in a composition comprising a solution that is isotonic to a biological sample, such as a buffer. In this aspect, a biological sample may be added to the plurality of detection agents, and optionally stored prior to the carrying out of the disclosed methods using the biological sample.

In some aspects, the methods may be carried out using reagents provided in the form of a kit, such as a package which houses a container which contains, for example, detection agents for one or more biomarkers, and instructions for carrying out one or more steps of the method, or interpreting the results based on the method. The kit may optionally also contain one or more therapeutic agents currently employed for treating a disease state as described herein. For example, a kit containing one or more compositions comprising active agents provided herein in combination with one or more additional active agents may be provided, or separate pharmaceutical compositions containing an active agent as provided herein and additional therapeutic agents may be provided. The kit may also contain separate doses of an active agent provided herein for serial or sequential administration. The kit may optionally contain one or more diagnostic tools and instructions for use. The kit may contain suitable delivery devices, e.g., syringes, and the like, along with instructions for administering the active agent(s) and any other therapeutic agent. The kit may optionally contain instructions for storage, reconstitution (if applicable), and administration of any or all therapeutic agents included. The kits may include a plurality of containers reflecting the number of administrations to be given to a subject or the number of biomarkers to be detected.

### Examples

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

In (pediatric) anti-TNF clinical trials, clinical remission at one year was only 55.8% 7 and 33.3% 8 with one real-world pediatric study showing endoscopic remission of 39%.⁹ Anti-TNF dose optimization following proactive therapeutic drug monitoring (TDM) in primary responders has been shown to improve rates of steroid-free clinical remission.¹⁰⁻¹²

With the recent expansion of second-line therapeutic options to manage CD, development of an anti-TNF companion diagnostic (predictive biomarker panel) is vital to adopt a personalized approach to drug selection. Unlike use of pharmacogenetics to tailor cancer management,¹³⁻¹⁵ pairing a patients immunophenotype with a biologic or small molecule has not yet occurred in real world clinical practice but is currently under study in a clinical trial. The primary driver to personalize drug selection is to quickly curtail ongoing intestinal inflammation and reduce CD-related complications as the 10 year rate of surgical resection in pediatric IBD is 38%.¹⁶ In addition, if a patient had a favorable immune profile to respond to anti-TNF, clinicians would be more apt to consider treatment optimizations (dose intensification or addition of combination therapy) during a CD flare (loss of response).

The clinical use of anti-TNF companion diagnostics are limited, with the most well referenced including blood biomarkers,¹⁷⁻¹⁹ and transcriptomics^{20,21} or single-cell analysis from intestinal tissue biopsies.²² While many factors have been studied to predict anti-TNF response, further validation studies are needed to fulfill all criteria to be classified as a prognostic indicator (Gisbert JCC 2020). In the largest CD prospective cohort study (PANTS) to date with 1241 anti-TNF naïve pediatric patients (>6 years old) receiving anti-TNF (infliximab or adalimumab) in the United Kingdom, low anti-TNF concentrations at the end of induction was the only independent factor associated with primary anti-TNF non-response²³ and may be the most commonly used biomarker in real world practice.

Small collections of plasma proteins have been associated anti-TNF response in CD,^{24,25} however, use of a large-scale, discovery proteomic platform has been mainly used to assess risk of developing CD. With the recognition of a primary non-response rate of 20-30% and 10-12% yearly loss of response rate despite anti-TNF dose optimization baseline predictors of primary anti-TNF response, improved methods for treating CD patients are needed.

Results

Patient Cohorts

The plasma proteome was assessed from 59 anti-TNF naïve CD subjects prior to starting infliximab and 19 healthy controls. The CD mean age of 13 (3.9) years, 32.2% female and 93.2% white race was similar to healthy controls (HC). Only one subject received combination therapy during induction, 57.6% were receiving prednisone at the start of infliximab and all 59 CD patients had a fCal>50 μg/g. Additional CD and HC baseline demographics and CD characteristics are reported in Table 1. Large scale proteomic analysis using the SOMAscan™ platform was performed on all baseline samples, but constrained to 50/59 and 26/59 at dose 4 and year 1, respectively.

Crohn's Disease Proteome

Starting with the 1304 proteins in the SOMAscan™ library, protein abundance between the 59 CD patients immediately prior to receiving the first dose of infliximab and the 19 HC was assessed. 725 were differentially abundant between CD and HC (FDR correction) with 78 proteins>2 fold-higher and 75 proteins 2-fold lower. To control for the 34 CD patients exposed to prednisone, 616 proteins were found to be differently expressed between CD and HC with 66 and 60 proteins 2-fold higher and 2-fold lower, respectively.

Baseline Proteome and Early Biochemical Nonresponse

Baseline protein abundance was evaluated between BioRem (n=12) and non-BioRem (n=36) at dose 4 starting with the 725 proteins differentially abundant in CD patients. 40 proteins (29 up, 11 down) were differently expressed with FCN3, CKM, SOST, TNFRSF11A and PRSS27 as the top 5 elevated proteins in non-remitters. Similarly, in the CD unexposed to prednisone, 74 proteins were differently abundant (38 up, 36 down) between dose 4 BioRem (n=7) and non-BioRem. The top 5 elevated proteins in biochemical remission (BioRem) were HBA1, FCN3, SH2D1A, TNFRSF11A and EPB41. Linear regression analysis was used to transform the 74 proteins into a single variable, principal component 1 (PC1) and then compared between dose 4 remitters and nonremitters.

Functional enrichment analysis with ToppGene was performed with the baseline proteins identified for dose 4 BioRem with proteins associated with decreased T-cell proliferation and TNF binding noticeably pathways identified. Remitters were enriched with TH17 cell differentiation and other signaling by interleukins.

The protein panel was further filtered with a minimal FC 2 to produce a more feasible companion diagnostic. This protein list was again transformed to a single variable with PC1 contributing xx % of the difference between BioRem and non-BioRem.

Start of Maintenance Proteome and Early Biochemical Nonresponse

WGCNA for Central CD Outcomes

The WGCNA framework was applied on proteomic assay samples from CD patients on infliximab at three different time points (baseline or visit 1, month 3 and month 12). Candidate protein modules were identified for each time point by correlating the modules with several different clinical and biochemical measurements and outcomes. From candidate modules the proteins in the top 10% percentile of the module membership score were considered as the intramodular hubs. Next, ToppGene tool Suite 37 was used to perform functional enrichment analysis for each of the candidate modules from each of the sample groups by using the respective module hubs.

Baseline Candidate Modules

To identify potential candidate proteins associated with primary non-response to infliximab as well as sustained remission, separate WGCNA runs were implemented on CD samples in the discovery cohort coming from three different time points (prior to starting IFX, prior to first maintenance dose and after one year of IFX treatment). The BL-M3 module (158 proteins) from baseline samples was strongly associated with multiple clinical and biochemical traits including wPCDAI score (both baseline and week14), fecal calprotein levels (at week14), and albumin levels (both baseline and week14) suggesting this module to be a potential “response” signature. The network hubs from the BL-M3 module (17 proteins) contained several macrophages (DSC2, TGFB1, TLR4, TNFRSF21) and fibroblast (IGFBP7, UNC5C, IL6ST, MATN2, NEGR1) markers and were enriched for cell projection morphogenesis process and extracellular matrix (ECM) pathways. Similarly, the BL-M6 module (119 proteins) was also strongly correlated with fecal calprotein levels along with CRP and nCD64 measurements at Week14, suggestive of a “non-response” signature. Functional analysis of the BL-M6 module hubs (13 proteins) indicated enrichments of EGF receptor signaling IL2-signaling and MAP Kinase signaling pathways. These hubs also contained marker proteins for Treg and native T-cells (TPT1, YWHAB, STAT3).

Early (Month 3) Candidate Modules

The Wk14-M6 module (94 proteins) from the week 14 samples showed strong correlations with fecal calprotein levels at both Week 14 and Week 26 but not so much at baseline or Week52. These hub proteins in the Wk14-M6 module (10 proteins) consisted of cell projection morphogenesis, BMP signaling, and ILEUM fibroblast markers (MATN2, FSTL1). Wk14-M7 (174 proteins) and Wk14-M4 (358 proteins) modules were identified which were not correlated with fecal calprotectin levels at Week14 but significantly associated at Week26 making them potential candidates for early “non-response”. Also, the Wk14-M2 module (114 proteins) was positively correlated with nCD64 levels at Week14 and the CRP levels at baseline.

Year 1 Candidate Modules

Finally, the Wk52-M6 module (158 proteins) among the year1 samples was positively correlated with the wPCDAI levels at Week 52 while being negatively correlated at Week 14 making it a potential candidate module involved in early-stage clinical remission. Functional analysis of the module hubs (19 proteins) showed enrichments for pathways and processes associated with cytokine response, immune response-regulation, abnormal serum protein physiology and regulation of membrane permeability. These hubs also contained marker proteins for ILEUM-inflamed macrophages (S100A9, S100A12, AIF1). The Wk52-M3 module (241 proteins) was found to be mildly correlated with fecal calprotein levels at Week 52 with the hub proteins (27 proteins) enriched for intestinal inflammation and abnormal inflammatory response phenotypes in mice. Conversely, the Wk52-M1 module (227 proteins) was moderately correlated with the wPCDAI levels at baseline and Week 14 but not at Week 52 making it a candidate for primary non-response. The Wk52-M1 hubs (28 proteins) contained ECM-related proteins (BMP6, GDF2, MASP1, MMP13, OMD, PLXNC1, FSTL1, IGFBP7, POSTN) and were primarily involved in ECM, axon development and cell projection morphogenesis. It also contained multiple ILEUM-inflamed fibroblasts and endothelial cell markers (GAS1, MRC2, POSTN, IGFBP7, FSTL1).

Protein Overlaps and Preservation Analysis Among Candidate Modules

Significant overlaps among the different candidate modules were observed across the three analyses. The BL-M3:Wk14-M6:Wk52-M1 modules shared 60 common proteins, with 23 of them being intramodular hubs in the respective modules including FLRT2, CNTN4, NOTCH1 and EPHB2 proteins. Similarly, 76 common proteins were found in BL-M6:Wk14-M2:Wk52-M6 modules with 38 hubs (MAPK3, ADRBK1, YWHAB, MAPKAPK2—hubs in all three) among them. Finally, 87 common proteins were observed among BL-M2:Wk14-M7:Wk52-M3 modules with most of them (41 proteins) being intramodular hubs. These common proteins also included 11 proteins (SPP1, CADM1, SEMA6A, SLITRK5, EFNB2, IDS, CD109, ENG, EPHA1, EFNA5, AMIGO2) which are module hubs in all three candidate modules. Next, the preservation status of the baseline WGCNA modules was tested among the Week 14 and Week 52 samples (independently). The BL-M6 and BL-M2 modules were strongly conserved among the Week14 and Week52 samples while the BL-M3 module was moderately conserved at both the timelines.

Candidate Proteins Associated with Clinical and Biochemical Outcomes

Next, to identify hub proteins strongly associated with the different clinical and biochemical outcomes, protein candidates that are intramodular hubs in all three timelines were combined. In total, 27 hub proteins correlated with fecal calprotein levels, wPCDAI levels or nCD64 measurements at either of the three timelines.

TABLE
“Early responder” and “Non-Responder” protein
biomarkers. Proteins having a “*” indicate biomarkers
for best-case predictive model for steroid free status at week 52.

| | | | | | |
|---|---|---|---|---|---|
| Candidate | Cor_fcal | Cor_fcal | Cor_wPCDAI | Cor_wPCDAI | Cor_nCD64 |
| Hub | Week 14 | Week 52 | Week 14 | Week 52 | Week 14 |

Early Responder Biomarkers

| | | | | | |
|---|---|---|---|---|---|
| ENG | −0.22 | −0.25 | 0.21 | −0.05 | 0.14 |
| CADM1 | −0.35 | −0.33 | 0.12 | −0.05 | −0.06 |
| EFNA5* | −0.34 | −0.41 | 0.05 | −0.01 | −0.24 |
| AMIGO2* | −0.26 | −0.46 | 0.04 | −0.05 | 0.03 |
| SEMA6A | −0.24 | −0.37 | 0.19 | 0.05 | −0.16 |
| EFNB2 | −0.25 | −0.39 | 0.12 | 0.05 | −0.11 |
| EPHA1 | −0.31 | −0.34 | 0.16 | 0.09 | −0.28 |
| SPP1 | −0.17 | −0.25 | 0.21 | −0.02 | 0.15 |
| SLITRK5* | −0.32 | −0.3 | 0.07 | −0.03 | −0.09 |
| CD109 | −0.27 | −0.33 | 0.09 | 0.02 | 0.14 |
| IDS | −0.19 | −0.24 | 0.11 | −0.04 | 0.27 |
| FLRT2 | −0.25 | −0.41 | −0.01 | 0.03 | −0.26 |
| NOTCH1 | −0.31 | −0.02 | −0.04 | −0.18 | −0.27 |
| EPHB2 | −0.37 | −0.09 | −0.2 | −0.1 | −0.4 |

Non- Responder Biomarkers

| | | | | | |
|---|---|---|---|---|---|
| MAPK3* | 0.16 | −0.36 | −0.07 | 0.31 | 0.35 |
| YWHAB | 0.29 | −0.19 | −0.06 | 0.35 | 0.4 |
| ADRBK1 | 0.15 | −0.3 | 0.001 | 0.48 | 0.33 |
| MAPKAPK2 | 0.3 | −0.2 | 0.07 | 0.39 | 0.37 |
| CRK* | −0.1 | −0.45 | 0.12 | 0.02 | 0.56 |
| KPNB1 | −0.05 | −0.47 | 0.1 | −0.02 | 0.55 |
| XPNPEP1 | −0.02 | −0.39 | 0.13 | 0.08 | 0.5 |
| UBEL2L3* | 0.15 | −0.04 | −0.26 | −0.03 | 0.63 |
| PDXP* | 0.13 | −0.01 | −0.28 | −0.07 | 0.61 |
| RPS6KA5* | 0.14 | −0.02 | −0.22 | −0.1 | 0.59 |
| PSMA6* | 0.15 | 0.13 | −0.23 | −0.09 | 0.64 |
| IL2RG | 0.15 | 0.1 | −0.24 | −0.08 | 0.61 |
| SOD1 | 0.09 | 0.1 | −0.26 | −0.07 | 0.66 |

Based on trends of these correlations, proteins are clustered into two categories: potential early responders (ENG, CADM1, EFNA5, AMIGO2, SEMA6A, EFNB2, EPHA1, SPP1, SLITRK5, CD109, IDS, FLRT2, NOTCH1, EPHB2) and non-responders (MAPK3, YWHAB, ADRBK1, MAPKAPK2, CRK, KPNB1, XPNPEP1, UBE2L3, PDXP, RPS6KA5, PSMA6, IL2RG, SOD1). These hubs proteins were further tested for their predictive ability of the relevant outcomes. L2-regularized logistic regression models were designed for each outcome by using stratified training (60% of all samples) and test data (40% of all samples) splits. Each model is trained on the training split using k-fold cross-validation (k=3) and evaluated on the test set. Precision-Recall curves were plotted based on the test set predictions and area under the curve (AUC) scores were used to evaluate the predictive ability of each model. The set of 27 consensus hub proteins (predictors), were found to accurately predict the steroid free status of patients at week 52 (AUC=0.93). In addition, there were moderately accurate (AUC=0.6) in predicting primary non-response status of a patient at month 3. They were also found to be associated with fecal calprotein levels (high or low) at both week 14 (AUC=0.92) and week 52 (AUC=0.78).

Specific candidate genes associated with each of the outcome variable were filtered by following model selection approaches. Specifically, using the 27 candidate hubs as the initial set of predictors, step-wise model selection mechanisms were employed to identify the best set of proteins to useful to predict a given out come variable. A criterion-based selection procedure was used where the best-case model is chosen based on the Akaike Information Criterion (AIC) value and the observed AUC values were used for validating the final model. Again, using stratified train and test data partitions, candidate proteins associated with each of the outcome variables were identified. The best-case predictive model for steroid free status at week 52 included 9 candidate hubs (MAPK3, CRK, SLITRK5, EFNA5, AMIGO2, PSMA6, UBE2L3, PDXP, RPS6KA5) and achieved an area under the curve (AUC) value of 0.85. Similarly, several candidate proteins predictive of fecal calprotein levels at different timelines were identified. 11 protein hubs (FLRT2, EPHB2, NOTCH1, ADRBK1, SPP1, CADM1, XPNPEP1, AMIGO2, PSMA6, PDXP, RPS6KA5) were associated with calprotein levels at week 14 with an AUC score of 0.62. Additionally, the predictive model for calprotein levels at week 52 consisted of 9 proteins (FLRT2, EPHB2, NOTCH1, MAPK3, ADRBK1, CADM1, CD109, SOD1, IL2RG) achieving an AUC score of 0.8. Candidate hubs FLRT2, EPHB2, NOTCH1, ADRBK1 and CADM1 were found to be associated with fecal calprotein levels at both week 14 and week 52. These proteins were further studied based on enriched functional terms representing the various biological mechanisms involving them.

Materials and Methods

Patient Recruitment

In this multicenter, observational study, children (>1 years old) and young adults (<22 years old) with CD starting infliximab were enrolled between July 2014-December 2020. Recruitment occurred at four pediatric centers including Cincinnati Children's Hospital Medical Center, Connecticut Children's Medical Center, Nationwide Children's Hospital, and the Medical College of Wisconsin. Study exclusions included patients with prior anti-TNF exposure, a diagnosis of ulcerative colitis or IBD-unspecified, an enteric infection in the past two weeks, presence of an ileostomy or a history of an intra-abdominal abscess or inflammatory mass. The study protocol was approved by the Institutional Review Board at all four participating medical centers. In addition, healthy controls (HC) were recruited from Cincinnati Children's Hospital Medical Center. HC were screened for functional gastrointestinal disorders (irritable bowel syndrome, functional dyspepsia, abdominal migraine, functional abdominal pain, and functional constipation using the Rome IV questionnaire for children and adolescents.²⁶ HC were only included if they did not meet criteria for a functional gastrointestinal disorder and the fecal calprotectin (fCal) was <50 μg/g.

As an observational study, infliximab dosing regimens (included the use of an immunomodulator) were at the discretion of the subjects' primary gastroenterologist. Baseline demographics, CD location and CD phenotype (using the Paris classification²⁷) were collected immediately prior to starting infliximab with clinical disease activity scores using the weighted Pediatric Crohn's disease activity index (wPCDAI)²⁸ were recorded at baseline and prior to each infusion. Briefly, the wPCDAI is calculated with the combination of patient-reported symptoms (abdominal pain, general well-being, stools per day and presence of extraintestinal manifestations) in combination with laboratory markers (erythrocyte sedimentation rate and serum albumin) and physical exam findings (weight and perirectal exam). with clinical remission defined by a wPCDAI<12.5.28 Longitudinal biospecimens including blood prior to each infusion and stool (baseline, first maintenance dose and year1) were collected for one year.

Response Measures

Clinical and biochemical outcomes were prospectively recorded from baseline to one year from the start of infliximab. The primary outcome, biochemical remission (BioRem), was defined as a fecal calprotectin (fCal)<250 μg/g while remaining off prednisone and without a need for CD-related surgery by the first maintenance dose (dose4) or at year1. Clinical remission (CRem) was defined by a wPCDAI<12.5 at dose4 or year1.²⁸ In a subset of patients who had a colonoscopy performed 9-15 months from the start of infliximab, the simple endoscopic score-CD (SES-CD)²⁹ was performed to assess endoscopic severity. The SES-CD is based on the assessment of five segments of the bowel with grading (0-3) of four parameters for the five segments with endoscopic healing (EH) defined as a SES-CD<3.²⁹ Secondary outcomes at dose4 included biochemical response (BioResp, >50% reduction in baseline fCal) and clinical response (CResp, >17.5 point improvement in baseline wPCDAI). Patients stopping infliximab prior to years or those who required surgery prior to dose4 or year1 were recorded as treatment failures while patients lost to follow up were excluded from the primary analysis. As an observational study, patients with drug intensifications (increasing the dose and/or shortening the interval) were not considered a treatment failure.

Proteomic Quantification

Plasma protein abundance was quantified using the SOMAscan® 1304 protein analytes (SomaLogic Inc., Boulder CO) and performed at the Genome Technology Access Center at Washington University School of Medicine (St. Louis, MO). Protein capture uses Slow Off-rate Modified Aptamer (SOMAmer™) reagents to detect proteins spanning eight logs of abundance (from femtomolar to micromolar) with a median coefficient variance (CV) of 5.1. SOMAmer™ reagents are selected against proteins in their native folded conformations and require an intact, tertiary protein structure for binding, thereby, reducing the rate of detecting unfolded or denatured (inactive) proteins. The SOMAscan® library includes both secreted intracellular and extracellular proteins. Protein quantification included a SOMAmer-plasma protein binding step followed by a series of partitioning and wash steps that convert relative protein concentrations into measurable nucleic acid signals that are then quantified by hybridization to custom DNA microarrays. The readout for each protein is in relative fluorescent units (RFU). To ensure protein stability, peripheral blood was collected in P100 collection tubes (BD Biosciences) and immediately stored at −80 degrees Celsius in 250 μl aliquots. Complete sample analysis occurred in five batches using the same 1304 protein analyte assay.

Additional Biomarker Assays

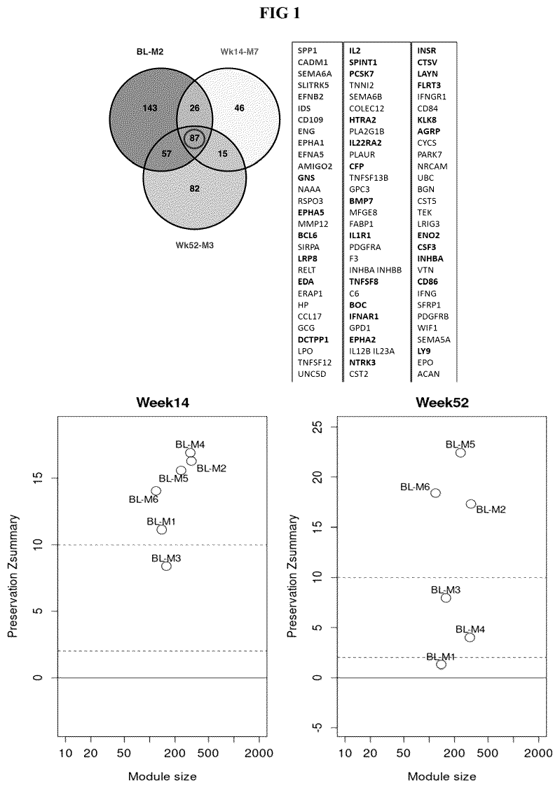

Infliximab and antibodies to infliximab (ATI) concentrations were measured with a drug-tolerant electrochemiluminescence immunoassay (Esoterix, LabCorp specialty lab, Calabasas, CA). With serial dilutions, the infliximab assay had an upper detection limit of 600 μg/mL, a lower detection limit of 0.4 μg/mL and an inter-assay coefficient of variation (CV) of 6.07-8.51%. The lower limit of ATI detection is 22 ng/mL. Fecal calprotectin was measured with an commercial ELISA kit (Buhlmann, Switzerland) with an inter-assay precision CV 6.6-14.5%.³⁰ Neutrophil surface expression of Fcγ Receptor 1 (CD64) is determined by a ratio of the mean fluorescence intensity (MFI) of granulocyte CD64 expression to the lymphocyte CD64 MFI by quantitative flow cytometry (FACSCantos, BD Biosciences, San Jose, CA) and reported as the neutrophil CD64 activity ratio (nCD64, further detailed in Supplementary FIG. 1). Cell populations are determined by forward and side scatter characteristics along with cell specific receptors; CD163 (monocytes) and CD45 (lymphocytes).

Statistical Analysis

Continuous variables are represented as means with standard deviations (SD) or medians with 25-75% interquartile range (IQR) depending on data distribution. Additional patient demographics and disease characteristics were described as frequencies (proportion). Logit regression was used to assess differences in protein abundance between CD and healthy controls and between CD remitters and non-remitters using baseline, month 3 and month 12 samples. To prevent type I errors, a false discovery rate (FDR) of <0.05 was applied. To identify protein and patient clusters, hierarchical clustering was performed using the Pearson Centered similarity measure and the Average linkage rule. Principal component analysis (PCA) was used to identify patient clusters with PCA plots generated. To further identify proteins associated between various outcomes, Venn diagrams were used to identify proteins that were consistent between the outcomes being assessed. The consistent proteins were submitted for ontological assessment through ToppCluster (toppcluster.cchmc.org). Finally, to determine the predictive capacity of the most relevant proteins, support vector machine analysis of significant, shared proteins with a more stringent statistical threshold (FC>2) was performed. Receiver operating characteristic (ROC) analysis was performed for individual proteins and protein sets with sensitivity, specificity, and the area under the ROC curve (AUROC) and 95% confidence intervals (CI) reported. Heatmaps were created to demonstrate results of the hierarchical clustering. Protein enrichment analysis was performed to establish pathways, biological processes, and molecular functions associated with the protein sets.

Weight Gene Co-Expression Analysis

As it was not feasible to perform endoscopic assessment on all enrolled patients, weighted gene co-expression analysis (WGCNA) was applied to further identify protein subsets that correlated with the multiple outcomes measures that were available. WGCNA is used to identify sub-networks or modules of co-expressed genes in whole transcriptomic or proteomic datasets.³¹ Correlating these modules with external clinical and phenotypic traits can help in identifying candidate genes or proteins and pathways associated with several diseases.³²⁻³⁶ Briefly, the WGCNA framework works by computing pairwise-correlations between protein abundance across all the samples in a study. These correlations are then raised to a higher power to retain only the strongly correlated proteins leading to a network of co-expressed proteins. These correlations are further used to compute topological overlaps between two proteins that indicate their relative interconnectedness within the co-expressed network. Finally, average linkage hierarchical clustering is applied on the matrix of topological similarities to identify the final protein clusters or modules. The WGCNA framework was applied at baseline, dose 4 and year 1 with candidate modules identified by correlating protein abundance with several biochemical measurements and clinical outcomes. From each candidate module, proteins in the top 10% of the module membership score were identified as intramodular hub proteins. Finally, ToppGene tool Suite³⁷ was used to perform functional enrichment analysis for each of the candidate modules from each of the sample groups by using the respective module hubs.

Statistical analyses were performed using GraphPad PRISM Version 7, R (Core Team, 2012) and SAS.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A method for treating intestinal inflammation and reducing Crohn's Disease (CD)-related complications in an individual in need thereof comprising
a. detecting one or more biomarker of a predictive biomarker panel in a biological sample of said individual;
b. determining whether said individual is likely to have a therapeutic response to an anti-TNF treatment based on said detecting of said one or more biomarker; and
c. administering one or both of an increased dosage of anti-TNF treatment and a combination therapy that includes an anti-TNF treatment to said individual determined to have said therapeutic response to said anti-TNF treatment.

2. The method of claim 1, wherein determining comprises detecting a biomarker selected from FCN3, CKM, SOST, TNFRSF11A, PRSS27, HBA1, FCN3, SH2D1A, TNFRSF11A, EPB41, and combinations thereof, said biomarker being a protein, peptide, variant, or fragment thereof.

3. The method of claim 1 wherein said one or more biomarker is selected from ENG, CADM1, EFNA5, AMIGO2, SEMA6A, EFNB2, EPHA1, SPP1, SLITRK5, CD109, IDS, FLRT2, NOTCH1, EPHB2, and combinations thereof, and wherein a deviation from a control value for said biomarker indicates that said individual is a responder.

4. The method of claim 1 wherein said one or more biomarker is selected from MAPK3, YWHAB, ADRBK1, MAPKAPK2, CRK, KPNB1, XPNPEP1, UBE2L3, PDXP, RPS6KA5, PSMA6, IL2RG, SOD1, and combinations thereof, and wherein a deviation from a control value for said biomarker indicates that said individual is a non-responder.

5. The method of claim 1, wherein said one or more biomarker comprises a biomarker panel, said biomarker panel comprising from about 5 to about 20 protein biomarkers, or from about 10 to about 15 protein biomarkers, selected from ENG, CADM1, EFNA5, AMIGO2, SEMA6A, EFNB2, EPHA1, SPP1, SLITRK5, CD109, IDS, FLRT2, NOTCH1, EPHB2, MAPK3, YWHAB, ADRBK1, MAPKAPK2, CRK, KPNB1, XPNPEP1, UBEL2L3, PDXP, RPS6KA5, PSMA6, IL2RG, and SOD1.

6. The method of claim 1, wherein said detecting is carried out by week 14 of treatment.

7. The method of claim 1, wherein said one or more biomarker comprises a first biomarker and a second biomarker, wherein said first biomarker is CD64.

8. The method of claim 1 wherein said anti-TNF treatment is selected from infliximab, adalimumab, and combinations thereof.

9. The method of claim 1, wherein said biological sample is selected from plasma, blood (venous or arterial), serum, urine, saliva, cerebrospinal fluid (CSF), synovial fluid, amniotic fluid, breast milk, sweat (eccrine or apocrine), nasal secretions, feces (stool), a tissue sample (e.g. bone marrow), or a combination thereof.

10. The method of claim 1, wherein said biological sample is a plasma sample.

11. The method of claim 1, wherein determining comprises detecting a two-fold change in said biomarker as compared to a control value.

12. The method of claim 11 wherein said control value is a level in a healthy control or a baseline level in said individual.

13. The method of claim 1 wherein said detecting is carried out via an immunoassay.

14. A plurality of detection agents for use as a companion diagnostic, comprising at least 2, or at least 3 or a least 4, or at least 5, or at least 6, or at least 7, or at least 8, or at least 9, or at least 10, or at least 11, or at least 12, or at least 13, or at least 14, or at least 15, or at least 16, or at least 17, or at least 18, or at least 19, or at least 20 detection agents, each detection agent specific for a biomarker selected from ENG, CADM1, EFNA5, AMIGO2, SEMA6A, EFNB2, EPHA1, SPP1, SLITRK5, CD109, IDS, FLRT2, NOTCH1, EPHB2, MAPK3, YWHAB, ADRBK1, MAPKAPK2, CRK, KPNB1, XPNPEP1, UBEL2L3, PDXP, RPS6KA5, PSMA6, IL2RG, and SOD1.

15. The plurality of detection agents of claim 14, said detection agents comprising a plurality of antibodies specific to said at least one biomarker.

16. The plurality of detection agents of claim 14, said detection agent being a nucleic acid specific to a gene expressing said at least one biomarker.

17. The plurality of detection agents of claim 14, said detection agent comprising a label for detection.

18. The plurality of detection agents of claim 17, said label being capable of quantification.

19. The plurality of detection agents of claim 18, said plurality of detection agents being provided in a composition comprising a solution that is isotonic to a biological sample.
